(19) United States
(12) Patent Application Publication
(10) Pub. No.: US 2024/0238578 A1
(43) Pub. Date: Jul. 18, 2024

(54) CONNECTOR ASSEMBLY WITH DISLODGEMENT PREVENTION

(71) Applicant: CareFusion 303, Inc., San Diego, CA (US)

(72) Inventors: Jason Andrew WINE, Brea, CA (US); George MANSOUR, Diamond Bar, CA (US)

(21) Appl. No.: 18/403,119

(22) Filed: Jan. 3, 2024

Related U.S. Application Data
(60) Provisional application No. 63/439,822, filed on Jan. 18, 2023.

(51) Int. Cl. A61M 39/10; A61M 25/00

(52) U.S. Cl. CPC A61M 39/1011; A61M 25/0014; A61M 2039/1066; A61M 2039/1083; A61M 2039/1088

(57) ABSTRACT

A connector assembly including a luer having a first end and second end opposite the second end, the luer having a luer body and a luer pin disposed within the luer body, and being configured to axially move relative to the luer pin, a catheter coupler having an opening and a valve, the catheter coupler configured to couple to the luer such that at least a portion of the luer pin is disposed through the opening and into the valve to form a fluid pathway between the luer and the catheter coupler, and a connector coupled to the luer such that the luer body is at least partially disposed within the connector. The connector having an engaged position and a disengaged position and configured to transition from the engaged position to the disengaged position in response to a pullout force.

### CROSS REFERENCE TO RELATED APPLICATION

This application claims the benefit of priority under 35 U.S.C. § 119 to U.S. Provisional Patent Application Ser. No. 63/439,822, entitled, CONNECTOR ASSEMBLY WITH DISLODGEMENT PREVENTION, filed on Jan. 18, 2023, the entire contents of which is incorporated herein by reference.

### FIELD OF THE INVENTION

The present disclosure generally relates to connectors, and, in particular, to connector assemblies having dislodgement prevention.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Often, tubing or catheters are coupled or secured to each other to allow fluid communication between various portions of tubing or catheters.

In some applications, such tubing or catheters may become dislodged due to improper securement and/or when the coupling is subject to forces greater than what the coupling is designed to withstand.

### SUMMARY

One or more embodiments of the present disclosure are directed to a connector assembly including a luer having a first end and second end opposite the second end, the luer having a luer body and a luer pin disposed within the luer body, the luer body being configured to axially move relative to the luer pin, wherein the luer has a central axis extending through the first end and the second end, a catheter coupler having an opening and a valve, the catheter coupler configured to couple to the luer such that at least a portion of the luer pin is disposed through the opening and into the valve to form a fluid pathway between the luer and the catheter coupler, and a connector coupled to the luer such that the luer body is at least partially disposed within the connector, the connector including an engaging arm and the connector having an engaged position and a disengaged position and is configured to transition from the engaged position to the disengaged position in response to a pullout force exceeding a predetermined threshold force. In the engaged position, the engaging arm is disposed proximate the central axis to secure the catheter coupler to the luer to prevent decoupling of the luer from the catheter coupler, and in the disengaged position, the engaging arm is pivoted away from the central axis to allow the luer to decouple from the catheter coupler.

In some embodiments, the pullout force exceeding the predetermined threshold force causes axial movement of the luer body relative to the luer pin along the central axis. The predetermined threshold force may be approximately 5 lbs. The pullout force exceeding the predetermined threshold force may cause a portion of the connector to axially move relative to the luer pin.

In some embodiments, the engaging arm includes a distal end proximate the first end of the luer, and in the engaged position, the distal end is proximate the central axis and in the disengaged position, the distal end is pivoted away from the central axis and away from the catheter coupler when the luer is coupled to the catheter coupler. The connector may include a biasing arm coupled to the engaging arm, the biasing arm configured to bias the distal end of the engaging arm to be proximate to the central axis.

In some embodiments, the connector includes a biasing arm coupled to the engaging arm and the luer body includes a protrusion, the protrusion disposed proximate the biasing arm and configured to apply a pushing force on the biasing arm when the pullout force exceeds the predetermined threshold force. In response to the pullout force exceeding the predetermined threshold force, the luer body axial may move relative to the luer pin causing axial movement of the protrusion.

In some embodiments, the connector assembly includes a housing having an opening and coupled to the luer such that the luer pin extends through the opening and the luer body is proximate the opening. The connector may include a base disposed opposite a distal end of the engaging arm, the base coupled to the housing. When the pullout force exceeds the predetermined threshold, the luer body may axially move relative to the base and the housing. The connector may include a connecting arm coupling the base to the engaging arm, the connecting arm having a contracted position and an expanded position and biased to be in the contracted position. In the engaged position the connecting arm may be in the contracted position and in the disengaged position, the connecting arm may be in the expanded position. In the disengaged position, the connecting arm may be in the expanded position and may apply a pulling force on the engaging arm causing the engaging arm to pivot away from the central axis and away from the catheter coupler when the luer is coupled to the catheter coupler.

In some embodiments, the luer includes a biasing element disposed between the luer body and the luer pin, the biasing element biasing the luer body towards the opening of the housing, luer body includes a protrusion extending from the luer body and the housing includes a cutout sized and shaped to receive the protrusion, the cutout having a raised indent such that in the engaged position, the protrusion is disposed between the raised indent and the cutout and in the disengaged position the raised indent is disposed between the protrusion and the cutout. The raised indent may increase the predetermined threshold force required for connector to transition from the engaged position to the disengaged position.

In some embodiments, the central axis bifurcates the luer, the connector, and the catheter coupler when the catheter coupler is coupled to the luer and the luer is at least partially disposed within the connector.

In some embodiments, in the disengaged position, the catheter coupler is configured to decouple from the luer causing the fluid pathway to be interrupted.

In some embodiments, the connector assembly of claim 1 further includes a cover disposed over the luer and the connector, the cover having a slot allowing access to the luer and the connector.

One or more embodiments of the present disclosure are directed to a connector assembly including a luer having a first end and second end opposite the second end, the luer having a luer body and a luer pin disposed within the luer body, the luer body being configured to axially move relative to the luer pin, wherein the luer has a central axis extending through the first end and the second end, wherein the luer includes a biasing element disposed between the luer body and the luer pin, a housing having an opening and coupled to the luer such that the luer pin extends through the opening and the luer body is proximate the opening, the biasing element biasing the luer body towards the opening, a catheter coupler having an opening and a valve, the catheter coupler configured to couple to the luer such that at least a portion of the luer pin is disposed through the opening and into the valve to form a fluid pathway between the luer and the catheter coupler, and a connector having a base coupled to the housing and a plurality of engaging arms coupled to the base via one or more connecting arms, the connector being coupled to the luer such that the luer body is at least partially disposed within the connector, the connector having an engaged position and a disengaged position and is configured to transition from the engaged position to the disengaged position in response to a pullout force exceeding a predetermined threshold force, the one or more connecting arms having a contracted position and an expanded position, wherein the connecting arms are biased to be in the contracted position. In the engaged position, the connecting arm is in the contracted position and the plurality of engaging arms are disposed proximate the central axis to secure the catheter coupler to the luer to prevent decoupling of the luer from the catheter coupler, and in the disengaged position, the one or more connecting arms are in the expanded position and the plurality of engaging arms are pivoted away from the central axis to allow the luer to decouple from the catheter coupler. The pullout force exceeding the predetermined threshold force causes axial movement of the luer body relative to the luer pin and the housing along the central axis.

One or more embodiments of the present disclosure are directed to a connector assembly including a luer having a first end and second end opposite the second end, the luer having a luer body and a luer pin disposed within the luer body, the luer body being configured to axially move relative to the luer pin, wherein the luer has a central axis extending through the first end and the second end, wherein the luer includes a biasing element disposed between the luer body and the luer pin and a protrusion extending from the luer body, a housing having an opening and coupled to the luer such that the luer pin extends through the opening and the luer body is proximate the opening, the biasing element biasing the luer body towards the opening, a catheter coupler having an opening and a valve, the catheter coupler configured to couple to the luer such that at least a portion of the luer pin is disposed through the opening and into the valve to form a fluid pathway between the luer and the catheter coupler, and a connector coupled to the luer such that the luer body is at least partially disposed within the connector. The connector includes a base coupled to the housing, a plurality of engaging arms each coupled to the base via a plurality of connecting arms, each of the plurality of connecting arms having a contracted position and an expanded, the plurality of connecting arms biased to be in the contracted position, and a plurality of biasing arms coupling the plurality of engaging arms together, the plurality of biasing arms configured to engage the one or more protrusions. The connector has an engaged position and a disengaged position and is configured to transition from the engaged position to the disengaged position in response to a pullout force exceeding a predetermined threshold force. In the engaged position, the plurality of engaging arms are disposed proximate the central axis to secure the catheter coupler to the luer to prevent decoupling of the luer from the catheter coupler, and in the disengaged position, the plurality of connecting arms are in the expanded position, the protrusions apply a pushing force to the plurality of biasing arms, and the plurality of engaging arms are pivoted away from the central axis to allow the luer to decouple from the catheter coupler. The pullout force exceeding the predetermined threshold force causes axial movement of the luer body relative to the luer pin and the housing along the central axis.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1A is a side view of a connector assembly and a catheter coupler, in accordance with various aspects of the present disclosure.

FIG. 1B is a front view of the connector assembly of FIG. 1A.

FIG. 2A is a side view of a connector assembly of FIG. 1A coupled to a housing and having a shield, in accordance with some embodiments of the present disclosure.

FIG. 3 is a side perspective view of a connector of the connector assembly of FIG. 1A.

FIG. 4A is a side perspective view of the connector assembly of FIG. 1A in the engaged position.

FIG. 4B is a side perspective view of the connector assembly of FIG. 1A in the disengaged position.

FIG. 5A is a perspective view of a connector assembly having a mechanical indent, in accordance with various aspects of the present disclosure.

FIG. 5B is a side view of the connector assembly of FIG. 5A.

FIG. 6A is a perspective view of a connector assembly having a cover, in accordance with various aspects of the present disclosure.

FIG. 6B is a front view of the connector assembly of FIG. 6A.

FIG. 6C is a top perspective view of the connector assembly of FIG. 6A.

FIG. 7A is a side view of the connector assembly decoupled from a catheter coupler, in accordance with various aspects of the present disclosure.

FIG. 7B is a side view of a connector assembly of FIG. 7A engaged with the catheter coupler.

### DETAILED DESCRIPTION

The disclosed connector assembly includes a connector, a luer coupled to the connector, and a catheter coupler coupled to the luer. In some embodiments, the luer extends through the connector. The connector includes a base configured to couple to a housing to prevent axial movement of the base relative to the housing. The connector includes a first engaging arm and a second engaging arm. The first engaging arm and the second engaging are coupled to the base via one or more connecting arms. The base of the connector is coupled to the housing such that it is axially fixed in place relative to the engaging arms and the connecting arms. The first engaging arm is coupled to the second engaging arm via one or more biasing arms. The engaging arms are configured to engage a catheter coupler. The catheter coupler is configured to be coupled to the luer such that the engaging arms couple to the external surface of the catheter coupler, securing the catheter coupler to the luer when the luer is coupled to or disposed within the catheter coupler. The engaging arms are configured to prevent inadvertent decoupling of the catheter coupler from the luer.

The engaging arms have an engaged position and a disengaged position. In the engaged position, the engaging arms are disposed proximate to a central axis of the connector assembly compared to when the engaging arms are in the disengaged position. In the engaged position, engaging arms are configured to prevent inadvertent or accidental decoupling of catheter coupler from luer when the catheter coupler is coupled to luer. The engaging arms are configured to engage the catheter coupler to prevent inadvertent axial movement of the catheter coupler relative to the connector. By preventing axial movement of the catheter coupler relative to the connector, the connector can prevent unintended or accidental dislodgement of the catheter coupler from the luer and the connector assembly. The engaging arms can be configured to move from the engaged position to the disengaged position to allow for the decoupling of the catheter coupler from the luer.

In the disengaged position, the engaging arms are disposed away from the central axis of the connector assembly compared to the engaged position. The engaging arms being in the disengaged position allows for the catheter coupler to be easily decoupled from the luer. The engaging arms are configured to transition from an engaged position to a disengaged position in response to a pullout force exceeding a predetermined threshold force. By allowing the catheter coupler to be decoupled from the luer in response to the pullout force exceeding the predetermined threshold force, the catheter coupler can be easily removed, if needed, by applying an increased pullout force. In some embodiments, when the pullout force does not exceed the predetermined threshold force, the catheter coupler remains secured to the luer via the engaging arms.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for case of understanding. Reference numbers may have letter suffixes appended to indicate separate instances of a common element while being referred to generically by the same number without a suffix letter.

While the following description is directed to the connection of medical fittings for the administration of medical fluid using the disclosed coupler, it is to be understood that this description is only an example of usage and does not limit the scope of the claims. Various aspects of the disclosed coupler may be used in any application where it is desirable to secure the connection of various tubing and fittings.

The disclosed connector assembly overcomes several challenges discovered with respect to certain conventional connector assemblies. One challenge with certain conventional connector assemblies is that certain conventional connector assemblies may be difficult to decouple from a catheter, which results in catheters being dislodged from a patient. Further, during use, certain conventional connector assemblies may be designed to release or dislodge in response to relatively low pullout forces. For example, certain conventional connector assemblies may release in response to pullout forces experienced during patients rolling over in bed, patients catching tubing or lines on bed rails, moving patients to a different bed, fidgeting by pediatric patients, and/or disoriented adult patients pulling out their lines. Indeed, the Association for Vascular Access (AVA) Annual Scientific Meeting in 2017 reported a 10% dislodgement rate for 1,000 patients fitted with peripheral IV catheters, translating to approximately 33 million dislodgements per year in the U.S. alone. Because the accidental or unintentional dislodgement of tubing, catheters, or fittings may interrupt the administration of medical fluids, the use of certain conventional connector assemblies is undesirable.

Therefore, in accordance with the present disclosure, it is advantageous to provide connector assemblies/couplers as described herein that allows for improved securement of fittings or connectors. The disclosed connector assemblies are structured as described herein so as to permit the secure retention of the connectors, while allowing intentional decoupling and removal of the connector assembly as required.

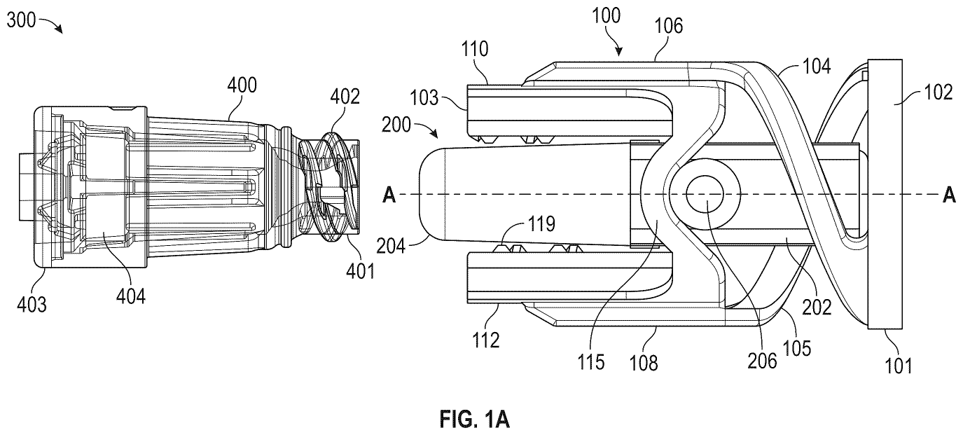

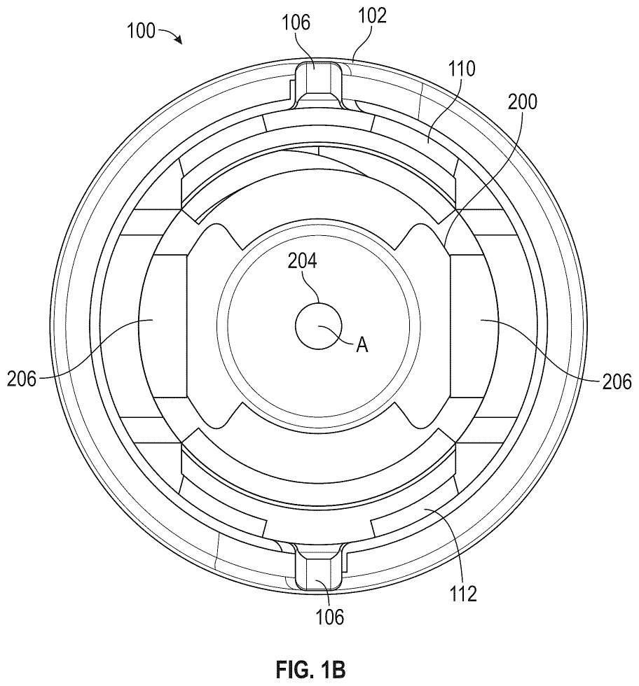

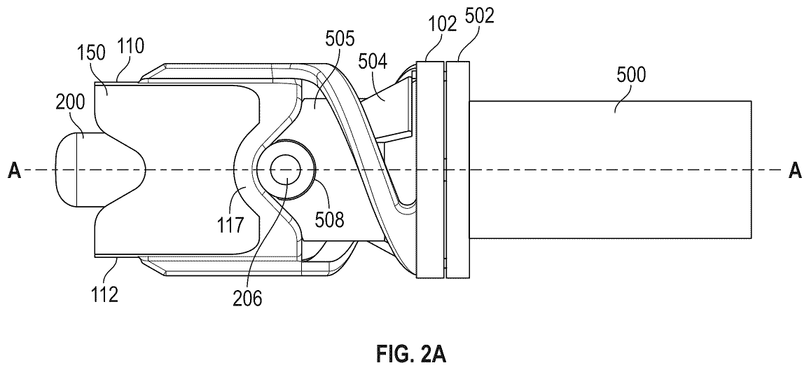

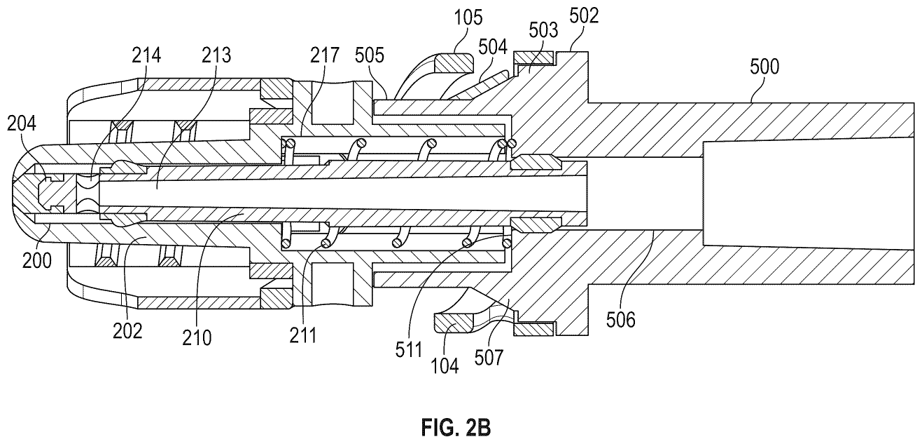

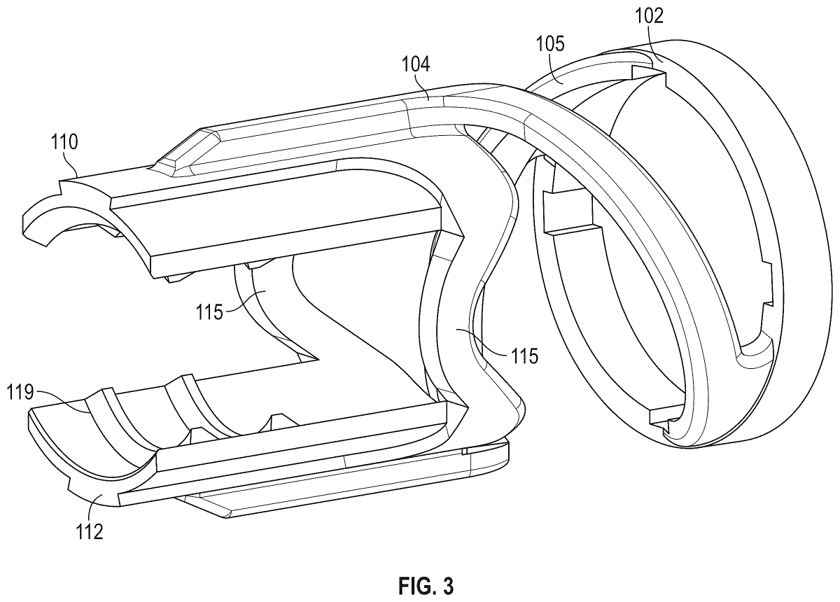

FIG. 1A is a side view of a connector assembly and a catheter coupler, in accordance with various aspects of the present disclosure. FIG. 1B is a front view of the connector assembly of FIG. 1A. FIG. 2A is a side view of a connector assembly of FIG. 1A coupled to a housing and having a shield, in accordance with some embodiments of the present disclosure. FIG. 3 is a side perspective view of a connector of the connector assembly of FIG. 1A.

Referring to FIGS. 1A-3, connector assembly 300 includes connector 100, luer 200, and catheter coupler 400. Connector assembly 300 allows for the flow of a fluid, such as a medical fluid, from a fluid source to a patient by releasably coupling a portion of tubing or line with another portion of tubing or line in fluid communication. In the depicted example, portions of tubing can be terminated with connectors/couplers, such as a connector 100, luer 200, and/or a catheter coupler 400. Connector 100, luer 200, and/or catheter coupler 400 can allow for the connection and/or disconnection of tubing to allow for selective fluid communication therebetween.

As illustrated, a first portion of tubing can be terminated by catheter coupler 400 to allow the first portion of tubing to be connected and/or disconnected to luer 200. Catheter coupler 400 may be configured to mate with luer 200. Catheter coupler 400 may be configured to couple with luer 200 to allow fluid communication between catheter coupler 400 and luer 200. In some embodiments, a first portion of tubing (e.g., tubing coupled to a catheter inserted in a patient) is coupled to catheter coupler 400, which is configured to couple to luer 200. Luer 200 may be configured to couple to a second portion of tubing (e.g., tubing coupled to a fluid source). In some embodiments, luer 200 is coupled to a first portion of tubing (e.g., tubing coupled to a catheter inserted in a patient) and catheter coupler 400 is coupled to a second portion of tubing (e.g., tubing coupled to a fluid source). Catheter coupler 400 coupling to luer 200 allows for first portion of tubing to be in fluid communication with the second portion of tubing. In some embodiments, when catheter coupler 400 is coupled to luer 200, fluid flows from a fluid source through the first portion of tubing, through luer 200 and catheter coupler 400 to the second portion of tubing and into the patient via a catheter.

In some embodiments, catheter coupler 400 is configured to receive at least of portion of luer 200. For example, a portion of luer 200 may be inserted into or disposed within catheter coupler 400 to allow catheter coupler 400 to be in fluid communication with luer 200. In some embodiments, connector 100 is configured to secure catheter coupler 400 to luer 200 when catheter coupler 400 is coupled to luer 200. When catheter coupler 400 is coupled to luer 200, connector 100 may be configured to secure catheter coupler 400 to prevent axial movement of catheter coupler 400 relative to luer 200.

In some embodiments, catheter coupler 400 includes first end 401 and second end 403. First end 401 may be opposite second end 403. First end 401 may be configured to receive luer 200. First end 401 of catheter coupler 400 may include opening 402. Opening 402 may be in fluid communication with tubing to allow fluid to pass through the catheter coupler 400. In some embodiments, the opening 402 is sized and shaped to receive luer 200. Catheter coupler 400 may include valve 404 configured to control the flow of fluid within catheter coupler 400. In some embodiments, valve 404 is at least partially disposed within opening 402. Valve 404 may be disposed proximate first end 401. In some embodiments, valve 404 is configured to receive or couple with luer 200 to allow catheter coupler 400 to be in fluid communication with luer 200. Second end 403 of catheter coupler 400 may be coupled to a catheter or needle via tubing. The catheter or needle may be inserted into a patient. In some embodiments, valve 404 extends from an area proximate first end 401 to an area proximate second end 403.

In some embodiments, connector assembly 300 includes luer 200. Luer 200 may be comprised of hard polymer such as polyvinyl chloride (PVC), acrylonitrile butadiene styrene (ABS), or any other type of hard polymer. Luer 200 may include luer body 202, luer pin 210 and opening 204. Luer 200 may have central axis A-A and luer pin 210 may extend at least a portion the length of luer 200 along central axis A-A. Luer pin 210 may be disposed within luer body 202 and may include channel 213. Channel 213 may be in fluid communication with opening 204. For example, when luer 200 couples to catheter coupler 400, fluid may flow from catheter coupler 400 through opening 204 and into channel 213. Luer 200 may be configured to allow for fluid communication with catheter coupler 400 when luer 200 is coupled to catheter coupler 400. For example, opening 402 of catheter coupler 400 may be configured to receive luer 200 such that luer 200 is inserted into opening 402 of catheter coupler 400, and catheter coupler 400 and luer 200 are in fluid communication. Alternatively, catheter coupler 400 may be inserted into opening 204 of luer 200. When luer 200 is coupled to catheter coupler 400, fluid may flow from a fluid source through luer pin 210 and opening 204 of luer 200 and through opening 402 of catheter coupler 400 to a patient.

In some embodiments, opening 402 of catheter coupler 400 and/or opening 204 of luer 200 include one or more features that allow for opening 402 to mate with opening 204. Opening 204 and/or opening 402 may be an inlet and/or an outlet. Opening 204 of luer 200 may fit together or otherwise engage with opening 402 of catheter coupler 400 to allow fluid communication between catheter coupler 400 and luer 200 and the portions of tubing coupled thereto. As can be appreciated, catheter coupler 400 and luer 200 can be coupled and decoupled to permit fluid communication as desired. As illustrated, opening 204 can include an outer portion that is smooth and otherwise free from threads. In some embodiments, opening 204 can include an outer portion that includes threads to facilitate coupling with catheter coupler 400. Catheter coupler 400 may couple with luer 200 to provide needle free connections. Advantageously, catheter coupler 400 can pair with luer 200 to form a leak-free closed system, allowing the delivery of various fluids, drugs, and/or liquids.

Luer 200 may be sized and shaped to mate with opening 402 of catheter coupler 400. In some embodiments, when luer 200 is coupled to catheter coupler 400, a fluid pathway is formed from a fluid source through luer 200 via opening 204 and through catheter coupler 400 via opening 402 to a catheter coupled to a patient. In some embodiments catheter coupler 400 couples to luer body 202 of luer 200. Catheter coupler 400 may couple to luer body 202 to allow opening 402 to be in fluid communication with luer pin 210 and channel 213. In some embodiments, luer pin 210 includes a valve (e.g., valve 214) configured to control the flow of fluid within luer 200. For example, valve 214 may be configured to control the flow of fluid within channel 213 of luer pin 210.

Referring to FIGS. 2A-2B, luer 200 may be coupled to housing 500. Housing 500 may be coupled to a portion of tubing, which may be coupled to a fluid source. Housing 500 may be comprised of hard polymer such as polyvinyl chloride (PVC), acrylonitrile butadiene styrene (ABS), or any other type of hard polymer. In some embodiments, housing 500 includes channel 506 which is in fluid communication with opening 204 and luer pin 210 of luer 200. Channel 506 may be configured to receive a portion of tubing or allow for fluid communication between the portion of tubing and luer 200. In some embodiments, housing 500 includes opening 511 configured to receive a portion of luer pin 210. At least a portion of luer pin 210 may be disposed within luer body such that luer body 202 abuts or contacts opening 511 of housing 500. Housing 500 may include channel 506, which may be in fluid communication with luer channel 213.

In some embodiments, channel 506 receives a portion of luer pin 210 and allows luer 200 to be in fluid communication with a portion of tubing. In some embodiments, luer pin 210 is disposed within luer body 202 and extends from luer body 202 (e.g., opposite opening 204) such that luer pin 210 extends into channel 506 of housing 500. Luer body 202 may contact, abut, or be disposed proximate opening 511 of channel 506. Luer pin 210 may be coupled to or partially disposed within channel 506 such that channel 506 is in fluid communication with luer pin 210. In some embodiments, luer pin 210 is at least partially secured within channel 506 such that axial movement of luer pin 210 relative to housing 500 is prevented.

In some embodiments, luer body 202 is configured to axially move relative to luer pin 210. Luer pin 210 may be coupled to housing 500 and may be at least partially disposed within luer body 202. In some embodiments, luer pin 210 is secured to housing 500 and luer body 202 is configured to axially move relative to luer pin 210 and/or housing 500. Luer 200 may include cavity 217 and may include biasing element 211 disposed within cavity 217. In some embodiments, at least a portion of luer pin 210 is disposed within cavity 217 of luer body 202. Biasing element 211 may be configured to bias luer body 202 towards housing 500. Biasing element 211 may include one end coupled to housing 500 (e.g., proximate opening 511) and another end coupled to luer body 202 (e.g., within cavity 217). In some embodiments, when catheter coupler 400 is coupled to luer 200 and is pulled axially away from housing 500, biasing element 211 may stretch (e.g., elongate) due to luer body 202 axially moving away from housing 500 relative to luer pin 210.

Luer 200 may include biasing element 211. Biasing element 211 may be disposed within the interior of luer body 202. In some embodiments, biasing element 211 is disposed between luer pin 210 and an internal surface of luer body 202. Biasing element 211 may be configured to allow for axial movement of luer body 202 relative to luer pin 210. Biasing element 211 may be configured to bias luer body 202 towards housing 500. Luer body 202 may axially move along central axis A-A relative to luer pin 210. For example, luer body 202 may axially move relative to luer pin 210 from abutting opening 511 of channel 506 to being away from opening 511. However, biasing element 211 may be configured to bias luer body 202 back to abutting opening 511 of channel 506 of housing 500.

Luer 200 may include one or more protrusions 206. In some embodiment, luer 200 includes two protrusion 206 disposed on luer body 202 of luer 200. Protrusions 206 may be disposed opposite one another. Luer 200 may include one, three, four, or greater than four protrusions 206. In some embodiments, protrusions 206 extend away from luer body 202 perpendicular to central axis A-A. Protrusions 206 may axially move due to axially movement of luer body 202. For example, luer body 202 may axially move away from housing 500 relative to luer pin 210, which may cause axial movement of protrusions 206 away from housing 500. Protrusion 206 may be substantially circular.

In some embodiments, movement of luer body 202 away from opening 511 and relative to luer pin 210 results in valve 214 within luer pin 210 closing to prevent leakage or spillage of fluid form luer pin 210 into luer body 202 or outside of luer 200. For example, when luer body 202 is pulled axially away from housing 500 (e.g., due to catheter coupler 400 being pulled), valve 214 may close luer pin 210 to prevent fluid from entering or existing luer 200.

In some embodiments, luer 200 includes a valve (e.g., valve 214) to allow for flow to pass therethrough when luer 200 is coupled to catheter coupler 400 and is configured to prevent or restrict flow when luer 200 is disconnected from catheter coupler 400. In some embodiments, luer 200 includes a sealing valve (e.g., valve 214) to seal the flow path between luer pin 210 and opening 204 when luer 200 is decoupled from catheter coupler 400. The sealing valve can be moved to an open position when catheter coupler 400 is coupled to luer 200 (e.g., in fluid communication with luer pin 210), allowing flow between luer 200 and catheter coupler 400.

Similarly, catheter coupler 400 may include a sealing valve (e.g., valve 404) to allow for flow to pass therethrough when catheter coupler 400 is coupled to luer 200 and is configured prevent or restrict flow when catheter coupler 400 is disconnected from luer 200. Catheter coupler 400 include a sealing valve (e.g., valve 404) to seal the flow out of opening 402 when catheter coupler 400 is decoupled from luer 200. Further, the sealing valve can be moved to an open position when luer 200 is coupled to catheter coupler 400, allowing flow between catheter coupler 400 and luer 200 (e.g., luer pin 210). In some embodiments, the sealing valve is formed from silicone.

In some embodiments, housing 500 includes ring 502, recessed collar 503, and one or more flaps 504. Recessed collar 503 may be disposed between ring 502 and flaps 504. In some embodiments, ring 502 is disposed proximate to channel 506 and/or opening 511 compared to flaps 504. Ring 502 may have a maximum diameter greater than the maximum diameter of recessed collar 503. Housing 500 may further include frustoconical or tapered portion 507 and body 505. Tapered portion 507 may be disposed between body 505 and recessed collar 503. Body 505 may extend from tapered portion 507 and may have a maximum diameter less than recessed collar 503. Tapered portion 507 may have a diameter that decreases from a portion proximate recessed collar 503 to a portion proximate body 505. In some embodiments, flaps 504 are disposed on body 505. A portion of luer 200 may be at least partially disposed within body 505 and may extend into channel 506. For example, a portion of luer pin 210 may be disposed within body 505. In some embodiments, housing 500 includes cutouts 508 sized and shaped to receive protrusions 206 of luer 200. Housing 500 may include any number of cutouts 508 corresponding to the number of protrusions 206 of luer 200.

In some embodiments, connector 100 includes base 102 and is coupled to housing 500 (e.g., via base 102). Recessed collar 503 may be configured to receive and secure base 102 of connector 100. For example, recessed collar 503 may be sized and shaped such that base 102 is able to be disposed around recessed collar 503 to secure base 102 to housing 500. In some embodiments, base 102 is secured to housing 500 by being disposed around recessed collar 503 and between flaps 504 and ring 502. For example, base 102 being disposed between ring 502 and flaps 504 may prevent axial movement (e.g., movement along central axis A-A) of base 102 relative to housing 500. In some embodiments, base 102 is secured to recessed collar 503 via an adhesive, magnet, hooks, or any other type of coupling element.

In some embodiments, a portion of luer 200 (e.g., luer pin 210) and a portion of connector 100 (e.g., base 102) are coupled to housing 500 such that luer 200 is disposed within connector 100. For example, a portion of luer 200 may be disposed within/coupled to housing 500 and base 102 of connector 100 may be coupled to housing 500 such that at least a portion of luer 200 (e.g., luer body 202) is extends through connector 100. Luer pin 210 may be coupled to housing 500 and luer body 202 may be configured to axially move relative to luer pin 210 and housing 500. In some embodiments, central axis A-A extends longitudinally along the length of luer 200 and housing 500 when a portion of luer 200 is coupled to housing 500. Connector 100 may be configured to at least partially radially surround luer 200 and central axis A-A.

Referring to FIGS. 1A-3, connector 100 may include first end 101 and second end 103. Central axis A-A may extend through first end 101 and second 103. In some embodiments, central axis A-A bifurcates luer 200 and connector 100. Connector 100 may be comprised a flexible material and may be comprised of a unitary structure. For example, connector 100 may be comprised of soft, flexible material, such as silicone or rubber. Connector 100 may include base 102 and may be configured to couple to housing 500 (e.g., via base 102) and extend away from housing 500. In some embodiments, a portion of connector 100 is configured to move axially along central axis A-A away and/or towards housing 500.

Connector 100 may include base 102, first connecting arm 104, second connecting arm 105, first engaging arm 106, and second engaging arm 108. Although FIGS. 1A-3 show connector 100 having two connecting arms and two engaging arms, connector 100 may have one, three, four, or greater than four connecting arms and/or engaging arms. Connecting arms 104, 105 may extend from base 102. In some embodiments, connecting arms 104, 105 are configured to move relative to base 102. For example, base 102 may be coupled to housing 500 and be fixed in place relative to connecting arms 104, 105. Connecting arms 104, 105 may be configured to contract towards base 102 and expand away from base 102. In some embodiments, connecting arm 104 is coupled to base 102 opposite from where connecting arm 105 couples to base 102. Connecting arms 104, 105 may be disposed between base 102, proximate to first end 101, and engaging arms 106, 108. First connecting arm 104 may be configured to extend from base 102 to couple to first engaging arm 106 and second connecting arm 105 may be configured to extend from base 102 and couple to second engaging arm 108. In some embodiments, first engaging arm 106 extends from first connecting arm 104 to an area proximate second end 103 and second engaging arm 108 extends from second connecting arm 106 to an area proximate second end 103. In some embodiments, first engaging arm 106 extends from first connecting arm 104 to be substantially parallel to second engaging arm 108, which extends from second connecting arm 105.

In some embodiments, first connecting arm 104 and second connecting arm 105 extend from base 102 in a spiral configuration. For example, first connecting arm 104 may spiral from base 102 in a counter-clockwise manner and second connecting arm 105 may extend from base 102 in a clockwise manner. First connecting arm 104 and second connecting arm 105 may be configured to bias first engaging arm 106 and second engaging arm 108 towards base 102. In some embodiments, first connecting arm 104 is configured to bias first engaging arm 104 towards base 102 and second connecting arm 105 is configured to bias second engaging arm 106 towards base 102. Engaging arms 106, 108 may be disposed between second end 103 and connecting arms 104, 105. Connecting arms 104, 105 may allow engaging arms 106, 108 to move axially along central axis A-A relative to base 102 and/or housing 500. For example, connecting arms 104, 105 may expand away from or contract toward base 102 resulting in movement of engaging arms 106, 108. Base 102, connecting arms 104, 105, and engaging arms 106, 108 may form a unitary structure.

In some embodiments, first connecting arm 104 couples to first engaging arm 106 at connection point 121 and second connecting arm 105 couples to second engaging arm 108 at connection point 123. First connection point 121 may be disposed on first engaging arm 106 proximate first end 101 and second connection point 123 may be disposed on second engaging arm 108 proximate second end 103. First engaging arm 106 may include first distal end 110, which may be opposite first connection point 121. In some embodiments, first distal end 110 is proximate first end 101. Second engaging arm 108 may include second distal end 112, which may be opposite second connection point 123. In some embodiments, second distal end 112 is proximate first end 101.

Connecting arms 104, 105 may be configured to be in a contracted state or in an expanded state. Connecting arms 104, 105 may be biased to be in the contracted position such that when connecting arms 104, 105 is in the expanded position, it applies a pulling force to engaging arms 106, 108. In the contracted state, first connecting arm 104 does not apply a pulling force to first engaging arm 106 at connection point 121 and second connecting arm 105 does not apply a pulling force to second engaging arms 108 at connection points 123. In the expanded state, first connecting arm 104 does applies a pulling force to first engaging arm 106 at connection point 121 and second connecting arm 105 does applies a pulling force to second engaging arms 108 at connection points 123.

In some embodiments, connector 100 includes one or more biasing arms 115. Biasing arm 115 may be configured to couple first engaging arm 106 to second engaging arm 108. Connector 100 may include two biasing arms 115 disposed opposite to one another. However, connector 100 may include any number of biasing arms 115. Biasing arms 115 may be biased to keep first engaging arm 106 substantially parallel to second engaging arm 108 and to keep first engaging arm 106 and second engaging arm 108 proximate to central axis A-A.

In some embodiments, upon axial movement of luer body 202 along central axis A-A away from housing 500, protrusions 206 abut and push against biasing arms 115. Protrusions 206 pushing against biasing arms 115 may cause connecting arms 104, 105 to expand (e.g., stretch out) away from base 102. For example, in response to a pullout force (e.g., force F) exceeding a predetermined threshold force, luer body 202 may axially move along central axis A-A away from housing 500 resulting in protrusions 206 applying a pushing force against biasing arms 115. Expansion of connecting arms 104, 105 may cause a pulling force to be applied to engaging arms 106, 108 pulling engaging arms 106, 108 towards base 102. In response to the pushing force of protrusions 206 against biasing arms 115 and the pulling force of connecting arms 104, 105 on engaging arms 106, 108, distal ends 110, 112 may be configured to move away from central axis A-A. In other words, engaging arms 106, 108 may pivot away from central axis A-A in response to protrusion 206 pushing against biasing arm 115 and connecting arms 104, 105 expanding and pulling engaging arms 106, 108 towards base 102.

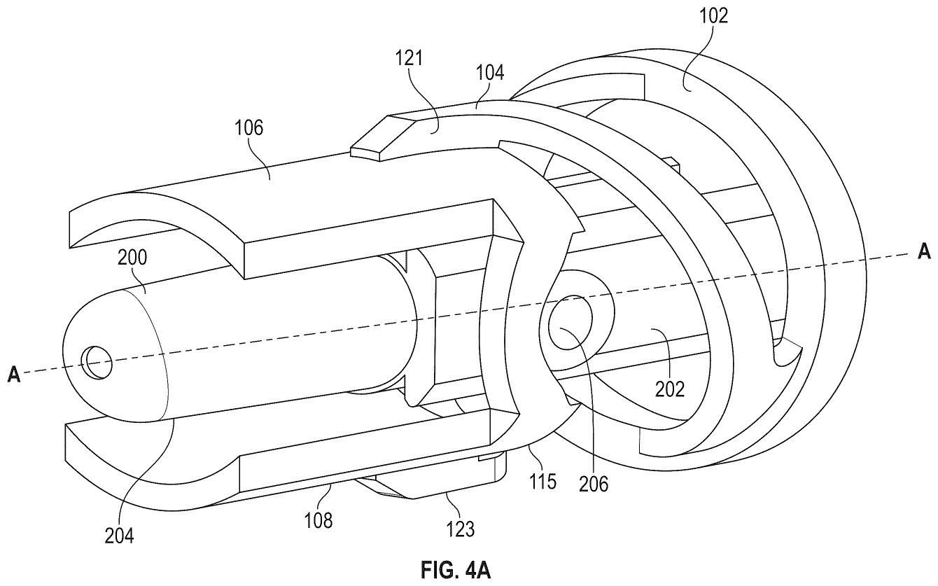

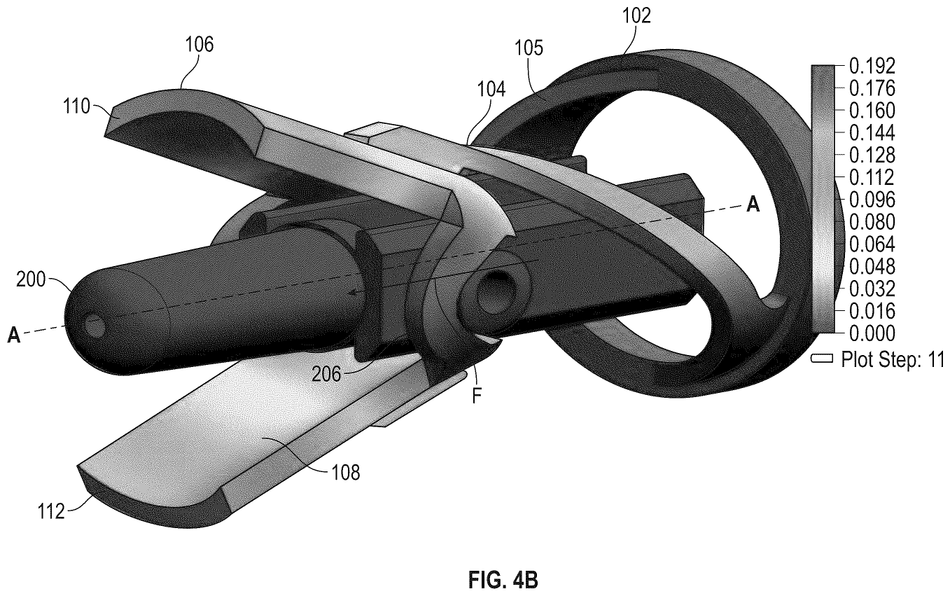

FIG. 4A is a side perspective view of the connector assembly of FIG. 1A in the engaged position and FIG. 4B is a side perspective view of the connector assembly of FIG. 2A in the disengaged position.

Referring to FIGS. 4A-4B, connector 100 may have an engaged position (FIG. 4A) and a disengaged position (FIG. 4B). In the engaged position, first distal end 110 of first engaging arm 106 and second distal end 112 of second engaging arm 108 may be disposed proximate central axis A-A and first engaging arm 106 may be disposed substantially parallel to second engaging arm 108. When connector 100 is in the engaged position and catheter coupler 400 is coupled to luer body 202, engaging arms 106, 108 may be configured to contact catheter coupler 400 and substantially prevent axial movement of catheter coupler 400 relative to luer body 202 and/or connector 100.

In some embodiments, engaging arms 106, 108 includes gripping features 119. Gripping features 119 may be configured to secure catheter coupler 400 to luer 200 when catheter coupler 400 is coupled to luer 200 and connector 100 is in the engaged position. In some embodiments, first engaging arm 106 and second engaging arm 108 each include one or more gripping features 119. Gripping features 119 prevent catheter coupler 400 from being decoupled from luer 200 when engaging arms 106, 108 are in the engaged position.

In the engaged position, connector 100 may prevent inadvertent decoupling of catheter coupler 400 from luer body 202 by contacting an external surface of catheter coupler 400 and preventing movement of catheter coupler 400 relative to luer body 202. When connector 100 is in the disengaged position, engaging arms 106, 108 may be disposed away from central axis A-A and away from luer body 202, resulting in engaging arms 106, 108 not contacting or abutting contact catheter coupler 400 when catheter coupler 400 is coupled to luer body 202. In the disengaged position, catheter coupler 400 may be easily decoupled from luer body 202.

Connector 100 may be configured to transition from the engaged position to the disengaged position in response to a pullout force exceeding a predetermined threshold force. For example, a pullout force (e.g., force F) may be applied to catheter coupler 400 when it is coupled to luer body 202. The pullout force may result in luer body 202 axially moving relative to house 500 due to catheter coupler 400 being coupled to luer body 202. In some embodiments, the pullout force (e.g., force F) results in luer body 202 axially moving relative to house 500 only when the pullout force exceeds the predetermined threshold force. When the pullout force does not exceed the predetermined threshold force, connector 100 may not transition from the engaged position to the disengaged position.

In some embodiments, connector 100 transitions from the engaged position (FIG. 4A) to the disengaged position (FIG. 4B) due to axial movement of luer body 202 in response to a pullout force (e.g., force F). Connector 100 may transition from the engaged position to the disengaged position when force F applied to luer body 202 (e.g., via catheter coupler 400) is greater than a predetermined threshold force. Luer 200 may be coupled to a fluid source via a first tube coupled to housing 500 (e.g., and luer pin 210) and luer body 202 may be coupled to catheter coupler 400, which may be coupled to a patient via a second tube. A force may be applied to the first tube, such as a pulling on tubing coupled to catheter coupler 400 and/or luer 200. The applied force may be transmitted to the patient if catheter coupler 400 remains coupled to luer body 202 and luer 200.

In some embodiments, decoupling of luer body 202 from catheter coupler 400 prevents the applied force from being transmitted to the patient and prevents inadvertent harm to the patient (e.g., pulling on a catheter or needle coupled to catheter coupler 400 and inserted into the patient). When the force, such as force F, exceeds a predetermined threshold force, connector 100 may be configured to cause catheter coupler 400 to decouple from luer 200 thereby preventing force F from being applied to the patient or a catheter/needle inserted into the patient.

FIG. 4A illustrates connector 100 being in the engaged position in response to force F being below or equal to the predetermined threshold force that is applied to luer body 202. For example, connector 100 may be subject to forces as a result of patients rolling over in bed, patients catching tubing or lines on bed rails, moving patients to a different bed, fidgeting by pediatric patients, and/or disoriented adult patients tugging on their lines. According to various embodiments of the present disclosure, when subject to lower forces (e.g., force F being below or equal to the predetermined threshold force), connector 100 may be configured to be in the engaged position so as to retain catheter coupler 400 and luer 200 together such that the fluid path remains open and fluid (e.g., IV fluid) may be administered from a fluid source (e.g., fluid bag, IV bag, fluid container, etc.) through luer 200 to catheter coupler 400 and the patient.

FIG. 4B depicts the disengaged position of connector 100 when the force F exceeds the predetermined threshold force that is applied to luer body 202. According to various embodiments of the present disclosure, when subject to higher forces (e.g., forces F exceeding the predetermined threshold force), connector 100 may be configured so as to move to the disengaged position resulting in the release or otherwise decoupling of catheter coupler 400 from luer body 202 at which point the fluid path between luer pin 210 and catheter coupler 400 may close and the medical fluid (e.g., IV fluid) may be discontinued from entering catheter coupler 400 and/or luer pin 210.

In some embodiments, the predetermined threshold force may be approximately 5 pounds (lbs). For example, if the patient coupled to catheter coupler 400, such as via a needle inserted into their skin, walks away from a fluid source or accidentally pulls on tubing coupled to the fluid source and the force exceeds 5 lbs, connector 100 may automatically move from the engaged position to the disengaged position thereby allowing catheter coupler 400 to release or decouple from luer body 202, effectively closing the fluid pathway connecting the patient to the fluid source (e.g., via catheter coupler 400 and luer 200). The predetermined threshold force may be from approximately 1 lbs to approximately 10 lbs, approximately 2 lbs to approximately 8 lbs, approximately 3 lbs to approximately 6 lbs, or greater than 10 lbs. Connector 100 causing the release or otherwise decoupling of catheter coupler 400 from luer body 202 is advantageous over conventional couplers, which may not result in release or decoupling thereby resulting bleeding, medication leaks, or other harm.

In some embodiments, force F exceeding the predetermined threshold force results in axial movement of luer body 202 relative to luer pin 210 and housing 500, which causes axial movement of protrusions 206 along central axis A-A. Force F exceeding the predetermined threshold force may result in axial movement of luer body 202 relative to luer pin 210, which may cause the valve within luer pin 210 to close and prevent any flow of fluid into or from luer pin 210. Axial movement of protrusions 206 may result in protrusions 206 abutting and applying a force (e.g., force F) to biasing arms 115. Axial movement of protrusions 206 against biasing arms 115 may also result in connecting arms 104, 105 applying a pulling force to engaging arms 106, 108 at connection points 121, 123, respectively.

The pulling force may cause biasing arms 115 to be pulled around the circumference of protrusion 206 resulting in engaging arms 106, 108 pivoting away from luer body 202 and central axis A-A. Connecting arms 104, 105 may apply a pulling force to engaging arms 106, 108, respectively, due to connecting arms 104, 105 being coupled to base 102, which is fixed relative to luer body 202. Therefore, as luer body 202 moves axially away from base 102, connecting arms 104, 105 transition from the contracted position to the expanded position resulting in a pulling force being applied to engaging arms 106, 108 at connection points 121, 123, respectively. The pulling force applied by connecting arms 104, 105 also results in biasing arms 115 stretching around protrusion 206, which causes engaging arms 106, 108 to pivot away from luer body 202 and central axis A-A. Engaging arms 106, 108 pivoting away from central axis A-A results in connector 100 moving from the engaged position to the disengaged position. The predetermined threshold force may be based on the flexibility or stiffness of biasing arms 115 and connecting arms 104, 105. For example, the higher the stiffness of biasing arms 115 and connecting arms 104, 105, the greater the predetermined threshold force.

In practice, catheter coupler 400 may be coupled to luer 200 and connecting arms 104, 105 may be in the contracted position resulting in engaging arms 106, 108 being in the engaged position and thus securing catheter coupler 400 to luer 200. Engaging arms 106, 108 may prevent inadvertent decoupling of catheter coupler 400 from luer 200. When a force (e.g., force F) is applied to luer 200, due catheter coupler 400 being coupled to luer body 202, that does not exceed the predetermined threshold force, connector 100 may remain in the engaged position. For example, minor forces (e.g., forces less than the predetermined threshold force) may be applied to luer 200 (or a tube coupled to luer 200). These minor forces may not result in connector 100 transitioning from the engaged position to the disengaged position since the force being applied by protrusions 206 to biasing arms 115 is not enough to transition connecting arms 104, 105 from the contracted state to the expanded state. This results in keeping catheter coupler 400 secured to luer body 202 via engaging arms 106, 108.

When force F being applied to luer 200 exceeds the predetermined threshold force, protrusion 206 may apply a force on biasing arms 115 resulting in connecting arms 104, 105 going from the contracted state to the expanded state thereby causing engaging arms 106, 108 to pivot away from central axis A-A causing connector 100 to go from the engaged position to the disengaged position and allowing catheter coupler 400 to decouple from luer 200. In some embodiments, biasing arm 115 of connector 100 is biased to press against protrusion 206 due to connecting arms 104, 105 being biased towards base 102 and base 102 being fixed relative to connecting arms 104, 105 and biasing arms 115.

Referring to FIG. 2A, connector 100 may include shield 150. Shield 150 may extend from first engaging arm 106 to second engaging arm 108. In some embodiments, shield 150 is coupled to biasing arm 115. Shield 150 may cover a portion of luer body 202 and may be configured to provide protection to luer body 202. In some embodiments, shield 150 is comprised of an over-molded elastomer.

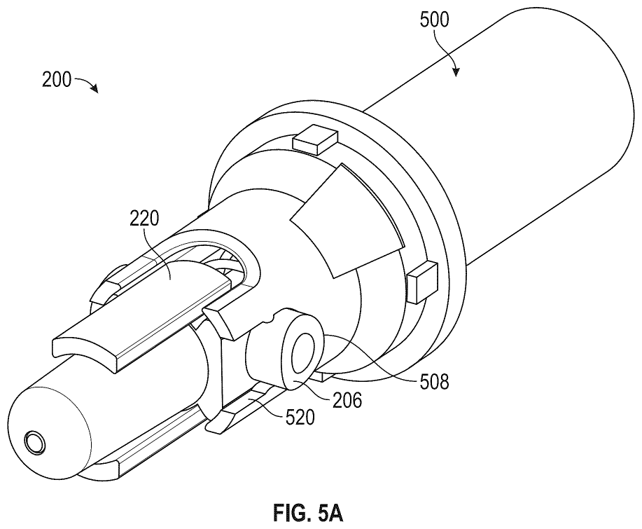

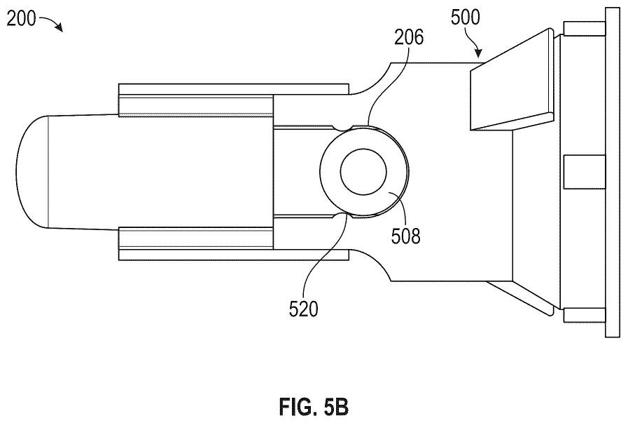

FIG. 5A is a perspective view of a connector assembly having a mechanical indent, in accordance with various aspects of the present disclosure. FIG. 5B is a side view of the coupling assembly of FIG. 5A.

Referring to FIGS. 5A-5B, housing 500 may include indent 520. Indent 520 may be a bump, ramp, or protrusion disposed on cutout 508. In some embodiments, protrusion 206 is disposed within cutout 508 and indent 520 is disposed proximate cutout 508. Indent 520 may be disposed distal to ring 502 compared to protrusion 206 when protrusion is disposed within cutout 508. Indent 520 may be configured to increase the force (e.g., force F) required to cause axial movement of protrusion 206 and thus cause connector 100 to transition from the engaged position to the disengaged position. In some embodiments, the presence of indent 520 increases the predetermined threshold force of force F that is needed to be exceeded to cause connector 100 to transition from the engaged position to the disengaged position. Housing 500 may include any number of indents 520. For example, housing 500 may include a total of one, two, three, four, or greater than four indents 520. In some embodiments, the greater the number of indents, the greater the predetermined threshold force of force F required to cause connector 100 to transition from the engaged position to the disengaged position.

In some embodiments, luer 200 includes securing arms 220. Securing arms 220 may be configured to assist with the coupling of catheter coupler 400 to luer 200. For example, when catheter coupler 400 is secured to luer 200, securing arms 220 may be configured to engage with or abut a portion of catheter coupler 400 to secure catheter coupler 400 to luer 200. In some embodiments, at least a portion of securing arms 220 pivots or moves towards central axis A-A when force F exceeds the predetermined threshold force, thereby allowing catheter coupler 400 to decouple from luer 200 when force F exceeds the predetermined threshold force.

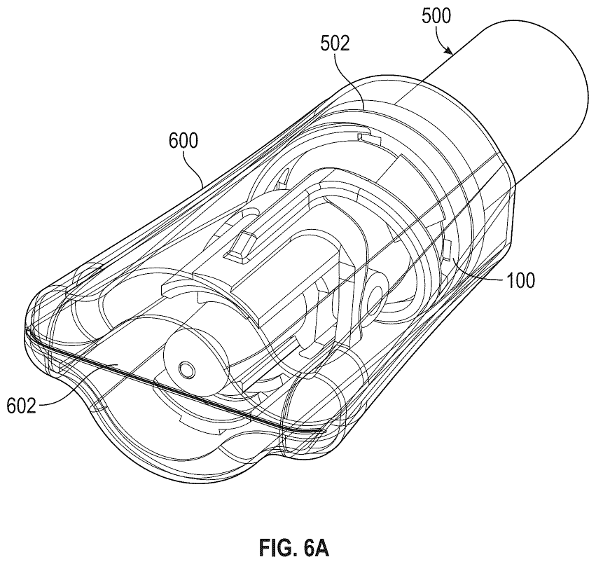

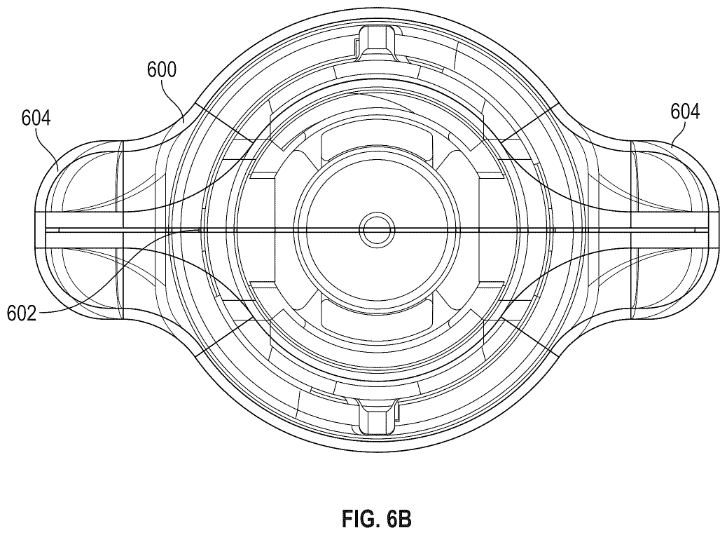

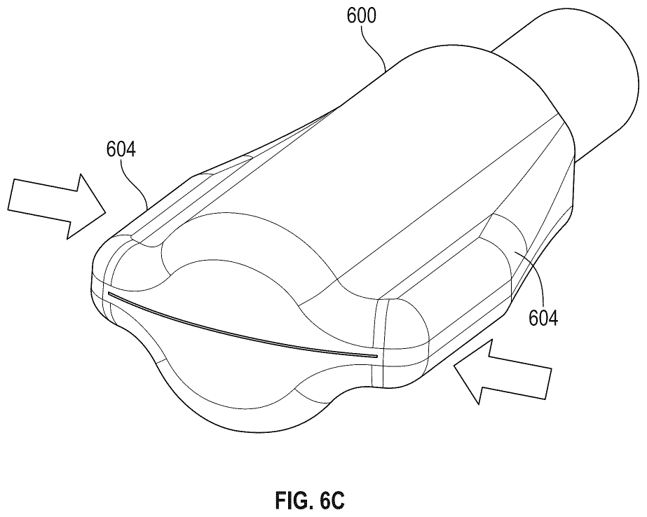

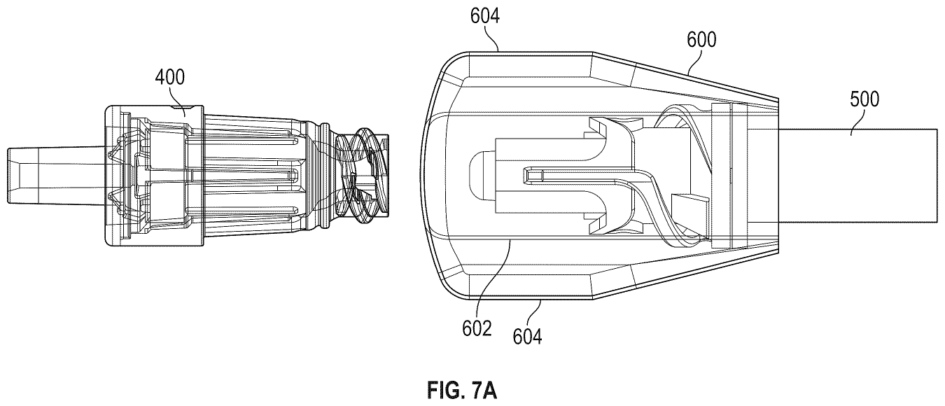

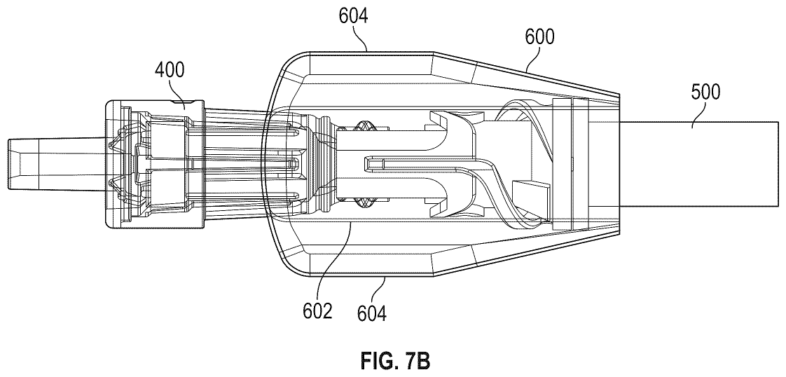

FIG. 6A is a perspective view of a connector assembly having a cover, in accordance with various aspects of the present disclosure. FIG. 6B is a front view of the connector assembly of FIG. 6A. FIG. 6C is a top perspective view of the connector assembly of FIG. 6A. FIG. 7A is a side view of a connector assembly having a cover engaged with a catheter, in accordance with various aspects of the present disclosure. FIG. 7B is a side view of the connector assembly of FIG. 7A disengaged with the catheter.

Referring to FIGS. 6A-7B, connector assembly 100 may include cover 600. Cover 600 may be configured to surround luer 200 and connector 100. In some embodiments, cover 600 couples to housing 500. In some embodiments, cover 600 couples to ring 502. Alternatively, cover 600 couples to any portion of housing 500. Cover 600 may include slot 602. Slot 602 may be configured to provide access to luer 200 and connector 100, which are disposed within cover 600.

In some embodiments, catheter coupler 400 is inserted through slot 602 to couple to luer 200 (FIG. 7A). Cover 600 may be waterproof and may prevent contaminants/debris from entering luer 200, such as via opening 204. In some embodiments, cover 600 includes flared portions 604, which are configured to squeezed (e.g., pressed towards luer 200) by a user to cause slot 602 to enlarge to provide access to luer 200 (FIG. 6C). A user may apply a force to flared portions 604 of cover 600 towards luer 200 to enlarge slot 602 thereby allowing catheter coupler 400 access into cover 600 for coupling to luer 200.

The disclosures described here including at least the following clauses:

Clause 1: A connector assembly comprising a luer having a first end and second end opposite the second end, the luer having a luer body and a luer pin disposed within the luer body, the luer body being configured to axially move relative to the luer pin, wherein the luer has a central axis extending through the first end and the second end, a catheter coupler having an opening and a valve, the catheter coupler configured to couple to the luer such that at least a portion of the luer pin is disposed through the opening and into the valve to form a fluid pathway between the luer and the catheter coupler, and a connector coupled to the luer such that the luer body is at least partially disposed within the connector, the connector including an engaging arm and the connector having an engaged position and a disengaged position and is configured to transition from the engaged position to the disengaged position in response to a pullout force exceeding a predetermined threshold force. In the engaged position the engaging arm is disposed proximate the central axis to secure the catheter coupler to the luer to prevent decoupling of the luer from the catheter coupler, and in the disengaged position, the engaging arm is pivoted away from the central axis to allow the luer to decouple from the catheter coupler.

Clause 2: The connector assembly of clause 1 further comprising a housing having an opening and coupled to the luer such that the luer pin extends through the opening and the luer body is proximate the opening.

Clause 3: The connector assembly of clause 2, wherein the connector includes a base disposed opposite a distal end of the engaging arm, the base coupled to the housing.

Clause 4: The connector assembly of clause 3, wherein the connector includes a connecting arm coupling the base to the engaging arm, the connecting arm having a contracted position and an expanded position and biased to be in the contracted position.

Clause 5: The connector assembly of clause 4, wherein in the engaged position the connecting arm is in the contracted position and in the disengaged position, the connecting arm is in the expanded position.

Clause 6: The connector assembly of clause 4, wherein, in the disengaged position, the connecting arm is in the expanded position and applies a pulling force on the engaging arm causing the engaging arm to pivot away from the central axis and away from the catheter coupler when the luer is coupled to the catheter coupler.

Clause 7: The connector assembly of clause 2, wherein the luer includes a biasing element disposed between the luer body and the luer pin, the biasing element biasing the luer body towards the opening of the housing.

Clause 8: The connector assembly of clause 2, wherein the luer body includes a protrusion extending from the luer body and the housing includes a cutout sized and shaped to receive the protrusion, the cutout having a raised indent such that in the engaged position, the protrusion is disposed between the raised indent and the cutout and in the disengaged position the raised indent is disposed between the protrusion and the cutout.

Clause 9: The connector assembly of clause 8, wherein the raised indent increases the predetermined threshold force required for connector to transition from the engaged position to the disengaged position.

Clause 10: The connector assembly of clause 1, wherein the engaging arm includes a distal end proximate the first end of the luer, and in the engaged position, the distal end is proximate the central axis and in the disengaged position, the distal end is pivoted away from the central axis and away from the catheter coupler when the luer is coupled to the catheter coupler.

Clause 11: The connector assembly of clause 10, wherein the connector includes a biasing arm coupled to the engaging arm, the biasing arm configured to bias the distal end of the engaging arm to be proximate to the central axis.

Clause 12: The connector assembly of clause 1, wherein the connector includes a biasing arm coupled to the engaging arm and the luer body includes a protrusion, the protrusion disposed proximate the biasing arm and configured to apply a pushing force on the biasing arm when the pullout force exceeds the predetermined threshold force.

Clause 13: The connector assembly of clause 12, wherein, in response to the pullout force exceeding the predetermined threshold force, the luer body axial moves relative to the luer pin causing axial movement of the protrusion.

Clause 14: The connector assembly of clause 1, wherein the pullout force exceeding the predetermined threshold force causes axial movement of the luer body relative to the luer pin along the central axis.

Clause 15: The connector assembly of clause 1, wherein the pullout force exceeding the predetermined threshold force causes a portion of the connector to axially move relative to the luer pin.

Clause 16: The connector assembly of clause 1, wherein the central axis bifurcates the luer, the connector, and the catheter coupler when the catheter coupler is coupled to the luer and the luer is at least partially disposed within the connector.

Clause 17: The connector assembly of clause 1, wherein in the disengaged position, the catheter coupler is configured to decouple from the luer causing the fluid pathway to be interrupted.

Clause 18: The connector assembly of clause 1 further comprising a cover disposed over the luer and the connector, the cover having a slot allowing access to the luer and the connector.

Clause 19: A connector assembly comprising a luer having a first end and second end opposite the second end, the luer having a luer body and a luer pin disposed within the luer body, the luer body being configured to axially move relative to the luer pin, wherein the luer has a central axis extending through the first end and the second end, wherein the luer includes a biasing element disposed between the luer body and the luer pin, a housing having an opening and coupled to the luer such that the luer pin extends through the opening and the luer body is proximate the opening, the biasing element biasing the luer body towards the opening, a catheter coupler having an opening and a valve, the catheter coupler configured to couple to the luer such that at least a portion of the luer pin is disposed through the opening and into the valve to form a fluid pathway between the luer and the catheter coupler, and a connector having a base coupled to the housing and a plurality of engaging arms coupled to the base via one or more connecting arms. The connector being coupled to the luer such that the luer body is at least partially disposed within the connector, the connector having an engaged position and a disengaged position and is configured to transition from the engaged position to the disengaged position in response to a pullout force exceeding a predetermined threshold force, the one or more connecting arms having a contracted position and an expanded position, wherein the connecting arms are biased to be in the contracted position. In the engaged position the connecting arm is in the contracted position and the plurality of engaging arms are disposed proximate the central axis to secure the catheter coupler to the luer to prevent decoupling of the luer from the catheter coupler, and in the disengaged position, the one or more connecting arms are in the expanded position and the plurality of engaging arms arc pivoted away from the central axis to allow the luer to decouple from the catheter coupler. The pullout force exceeding the predetermined threshold force causes axial movement of the luer body relative to the luer pin and the housing along the central axis.

Clause 20: A connector assembly comprising a luer having a first end and second end opposite the second end, the luer having a luer body and a luer pin disposed within the luer body, the luer body being configured to axially move relative to the luer pin, wherein the luer has a central axis extending through the first end and the second end, wherein the luer includes a biasing element disposed between the luer body and the luer pin and a protrusion extending from the luer body, a housing having an opening and coupled to the luer such that the luer pin extends through the opening and the luer body is proximate the opening, the biasing element biasing the luer body towards the opening, a catheter coupler having an opening and a valve, the catheter coupler configured to couple to the luer such that at least a portion of the luer pin is disposed through the opening and into the valve to form a fluid pathway between the luer and the catheter coupler; and a connector coupled to the luer such that the luer body is at least partially disposed within the connector. The connector comprising a base coupled to the housing, a plurality of engaging arms each coupled to the base via a plurality of connecting arms, each of the plurality of connecting arms having a contracted position and an expanded, the plurality of connecting arms biased to be in the contracted position, and a plurality of biasing arms coupling the plurality of engaging arms together, the plurality of biasing arms configured to engage the one or more protrusions. The connector has an engaged position and a disengaged position and is configured to transition from the engaged position to the disengaged position in response to a pullout force exceeding a predetermined threshold force. In the engaged position, the plurality of engaging arms are disposed proximate the central axis to secure the catheter coupler to the luer to prevent decoupling of the luer from the catheter coupler, and in the disengaged position, the plurality of connecting arms are in the expanded position, the protrusions apply a pushing force to the plurality of biasing arms, and the plurality of engaging arms are pivoted away from the central axis to allow the luer to decouple from the catheter coupler. The pullout force exceeding the predetermined threshold force causes axial movement of the luer body relative to the luer pin and the housing along the central axis.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A connector assembly comprising:
a luer having a first end and second end opposite the second end, the luer having a luer body and a luer pin disposed within the luer body, the luer body being configured to axially move relative to the luer pin, wherein the luer has a central axis extending through the first end and the second end;
a catheter coupler having an opening and a valve, the catheter coupler configured to couple to the luer such that at least a portion of the luer pin is disposed through the opening and into the valve to form a fluid pathway between the luer and the catheter coupler; and
a connector coupled to the luer such that the luer body is at least partially disposed within the connector, the connector including an engaging arm and the connector having an engaged position and a disengaged position and is configured to transition from the engaged position to the disengaged position in response to a pullout force exceeding a predetermined threshold force,
wherein in the engaged position the engaging arm is disposed proximate the central axis to secure the catheter coupler to the luer to prevent decoupling of the luer from the catheter coupler, and in the disengaged position, the engaging arm is pivoted away from the central axis to allow the luer to decouple from the catheter coupler.

2. The connector assembly of claim 1 further comprising:
a housing having an opening and coupled to the luer such that the luer pin extends through the opening and the luer body is proximate the opening.

3. The connector assembly of claim 2, wherein the connector includes a base disposed opposite a distal end of the engaging arm, the base coupled to the housing.

4. The connector assembly of claim 3, wherein the connector includes a connecting arm coupling the base to the engaging arm, the connecting arm having a contracted position and an expanded position and biased to be in the contracted position.

5. The connector assembly of claim 4, wherein in the engaged position the connecting arm is in the contracted position and in the disengaged position, the connecting arm is in the expanded position.

6. The connector assembly of claim 4, wherein, in the disengaged position, the connecting arm is in the expanded position and applies a pulling force on the engaging arm causing the engaging arm to pivot away from the central axis and away from the catheter coupler when the luer is coupled to the catheter coupler.

7. The connector assembly of claim 2, wherein the luer includes a biasing element disposed between the luer body and the luer pin, the biasing element biasing the luer body towards the opening of the housing.

8. The connector assembly of claim 2, wherein the luer body includes a protrusion extending from the luer body and the housing includes a cutout sized and shaped to receive the protrusion, the cutout having a raised indent such that in the engaged position, the protrusion is disposed between the raised indent and the cutout and in the disengaged position the raised indent is disposed between the protrusion and the cutout.

9. The connector assembly of claim 8, wherein the raised indent increases the predetermined threshold force required for connector to transition from the engaged position to the disengaged position.

10. The connector assembly of claim 1, wherein the engaging arm includes a distal end proximate the first end of the luer, and in the engaged position, the distal end is proximate the central axis and in the disengaged position, the distal end is pivoted away from the central axis and away from the catheter coupler when the luer is coupled to the catheter coupler.

11. The connector assembly of claim 10, wherein the connector includes a biasing arm coupled to the engaging arm, the biasing arm configured to bias the distal end of the engaging arm to be proximate to the central axis.

12. The connector assembly of claim 1, wherein the connector includes a biasing arm coupled to the engaging arm and the luer body includes a protrusion, the protrusion disposed proximate the biasing arm and configured to apply a pushing force on the biasing arm when the pullout force exceeds the predetermined threshold force.

13. The connector assembly of claim 12, wherein, in response to the pullout force exceeding the predetermined threshold force, the luer body axial moves relative to the luer pin causing axial movement of the protrusion.

14. The connector assembly of claim 1, wherein the pullout force exceeding the predetermined threshold force causes axial movement of the luer body relative to the luer pin along the central axis.

15. The connector assembly of claim 1, wherein the pullout force exceeding the predetermined threshold force causes a portion of the connector to axially move relative to the luer pin.

16. The connector assembly of claim 1, wherein the central axis bifurcates the luer, the connector, and the catheter coupler when the catheter coupler is coupled to the luer and the luer is at least partially disposed within the connector.

17. The connector assembly of claim 1, wherein in the disengaged position, the catheter coupler is configured to decouple from the luer causing the fluid pathway to be interrupted.

18. The connector assembly of claim 1 further comprising:
a cover disposed over the luer and the connector, the cover having a slot allowing access to the luer and the connector.

19. A connector assembly comprising:
a luer having a first end and second end opposite the second end, the luer having a luer body and a luer pin disposed within the luer body, the luer body being configured to axially move relative to the luer pin, wherein the luer has a central axis extending through the first end and the second end, wherein the luer includes a biasing element disposed between the luer body and the luer pin;
a housing having an opening and coupled to the luer such that the luer pin extends through the opening and the luer body is proximate the opening, the biasing element biasing the luer body towards the opening;
a catheter coupler having an opening and a valve, the catheter coupler configured to couple to the luer such that at least a portion of the luer pin is disposed through the opening and into the valve to form a fluid pathway between the luer and the catheter coupler; and
a connector having a base coupled to the housing and a plurality of engaging arms coupled to the base via one or more connecting arms, the connector being coupled to the luer such that the luer body is at least partially disposed within the connector, the connector having an engaged position and a disengaged position and is configured to transition from the engaged position to the disengaged position in response to a pullout force exceeding a predetermined threshold force, the one or more connecting arms having a contracted position and an expanded position, wherein the connecting arms are biased to be in the contracted position,
wherein in the engaged position the connecting arm is in the contracted position and the plurality of engaging arms are disposed proximate the central axis to secure the catheter coupler to the luer to prevent decoupling of the luer from the catheter coupler, and in the disengaged position, the one or more connecting arms are in the expanded position and the plurality of engaging arms are pivoted away from the central axis to allow the luer to decouple from the catheter coupler,
wherein the pullout force exceeding the predetermined threshold force causes axial movement of the luer body relative to the luer pin and the housing along the central axis.

20. A connector assembly comprising:
a luer having a first end and second end opposite the second end, the luer having a luer body and a luer pin disposed within the luer body, the luer body being configured to axially move relative to the luer pin, wherein the luer has a central axis extending through the first end and the second end, wherein the luer includes a biasing element disposed between the luer body and the luer pin and a protrusion extending from the luer body;
a housing having an opening and coupled to the luer such that the luer pin extends through the opening and the luer body is proximate the opening, the biasing element biasing the luer body towards the opening;
a catheter coupler having an opening and a valve, the catheter coupler configured to couple to the luer such that at least a portion of the luer pin is disposed through the opening and into the valve to form a fluid pathway between the luer and the catheter coupler; and
a connector coupled to the luer such that the luer body is at least partially disposed within the connector, the connector comprising:
a base coupled to the housing;
a plurality of engaging arms each coupled to the base via a plurality of connecting arms, each of the plurality of connecting arms having a contracted position and an expanded, the plurality of connecting arms biased to be in the contracted position; and
a plurality of biasing arms coupling the plurality of engaging arms together, the plurality of biasing arms configured to engage the one or more protrusions,
wherein the connector has an engaged position and a disengaged position and is configured to transition from the engaged position to the disengaged position in response to a pullout force exceeding a predetermined threshold force,
wherein in the engaged position, the plurality of engaging arms are disposed proximate the central axis to secure the catheter coupler to the luer to prevent decoupling of the luer from the catheter coupler, and in the disengaged position, the plurality of connecting arms are in the expanded position, the protrusions apply a pushing force to the plurality of biasing arms, and the plurality of engaging arms are pivoted away from the central axis to allow the luer to decouple from the catheter coupler,
wherein the pullout force exceeding the predetermined threshold force causes axial movement of the luer body relative to the luer pin and the housing along the central axis.
